(19) United States
(12) Patent Application Publication
(10) Pub. No.: US 2006/0089329 A1
(43) Pub. Date: Apr. 27, 2006

(54) Ready-to-use gemcitabine solution concentrates

(76) Inventors: Edgar Schridde, Hannover (DE); Bernd Merbach, Burgwedel (DE); Stefan-Peter Gimmel, Hannover (DE)

Correspondence Address: KENYON & KENYON LLP, ONE BROADWAY, NEW YORK, NY, 10004, US

(21) Appl. No.: 10/972,042

(22) Filed: Oct. 22, 2004

(51) Int. Cl. A61K 31/7072

(52) U.S. Cl. 514/49

(57) ABSTRACT

Provided are ready-to-use pharmaceutical compositions in the form of gemcitabine solution concentrates.

### FIELD OF THE INVENTION

The present invention relates to pharmaceutical preparations of gemcitabine in the form of ready-to-use solution concentrates.

### BACKGROUND OF THE INVENTION

Gemcitabine (2′-deoxy-2′,2′-difluorocytidine; 1-(4-amino-2-oxo-1H-pyrimidin-1-yl)-2-deoxy-2,2-difluororibose; dFdC; CAS No. 95058-81-4; C₉H₁₁F₂N₃O₄, Mᵣ 263.2) is an officially monographed substance in the Pharmacopoeia (Official Monographs, USP 27, 1st Supplement USP-NF, page 3060-61, relating to “Gemcitabine Hydrochloride” and “Gemcitabine for Injection”). Gemcitabine has the following chemical structure:

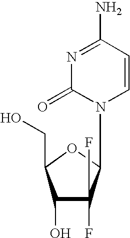

Gemcitabine is used in the treatment of viral infections or immunosuppressive therapy of autoimmune diseases. Gemcitabine was first disclosed in U.S. Pat. No. 4,526,988. U.S. Pat. No. 5,464,826 discloses the antineoplastic effectiveness of gemcitabine. Gemcitabine can be used therapeutically by itself, or in combination with other cytostatic drugs, such as with cisplatin in the treatment of local, advanced, or metastasized non-small-cell bronchial carcinoma, as well as advanced adenocarcinoma or cystadenocarcinoma of the exocrine pancreas.

The recommended dose for gemcitabine therapy is 1 g/m² of body surface area. As other nucleoside analogs, gemcitabine can also be used cytostatically in the therapeutic treatment of the most varied types of cancer, such as lymphatic or myeloid leukemia. Here, the administration of gemcitabine to treat the most varied cancer conditions is effected intravenously, in which case the active substance must be in the form of a solution

The gemcitabine preparations required for parenteral administration are currently available only in the form of lyophilisates, which must be reconstituted before administration to the patient. However, the use of such freeze-dried preparations has considerable disadvantages. First of all, the process of preparing these lyophilisates is complicated and costly. Secondly, reconstitution requires additional working steps and entails undesirable risks for the personnel involved. In particular, reconstitution of drug solutions from a dry substance can result in what has been called the “spray-back effect,” which may result in further contamination and risk to the personnel. Accordingly, in both the preparation of the lyophilisate and its reconstitution, any contamination of the personnel or stock with the highly effective cytostatic must be avoided. Furthermore, other errors in the handling of these lyophilisates can lead to serious problems in the treatment with gemcitabine, such as deviation in the concentration of the active substance, or microbial contamination of the solution prepared from the lyophilisate.

Moreover, known gemcitabine preparations reconstituted from lyophilisates have the disadvantage in that the gemcitabine concentration is limited to 38 mg/ml, and that such concentration can be achieved only at a pH of 2.7 to 3.3. This is known to be due to the fact that the solubility of gemcitabine in water decreases with increasing pH, so that its solubility is about 38 mg/ml within a pH range of 2.7 to 3.3, 16.0 mg/ml at a pH of 5, 15.3 mg/ml at a pH of 7, and 15.8 mg/ml at a pH of 9. In order to reduce the solution volume to be freeze-dried for the lyophilisate, the active substance is therefore dissolved in water at a pH range of 2.7 to 3.3. According to the directions for use or the technical information of GEMZAR® lyophilisate, the pH of solutions reconstituted from the GEMZAR® lyophilisate with NaCl solution is maintained precisely in this range of maximum solubility (pH of 2.7 to 3.3).

Furthermore, in order to maintain the recommended dose of 1 g/m² body surface area during gemcitabine therapy, large solution volumes must be used to reconstitute infusion solutions from the lyophilisates. For example, at an average dose of 1.8 g/1.8 m² body surface area, a reconstituted solution volume of 47 ml is diluted with 250 to 500 ml of a carrier solution. For reliable handling, it is advantageous to work with the smallest possible solution volumes. It is also advantageous in terms of operating costs to use the smallest possible packaging sizes (e.g., perforable stopper vials) for drug storage. These additional advantages are afforded by a ready-to-use solvent concentrate.

The object of the present invention is to make available ready-to-use gemcitabine solutions that do not entail the above-discussed risks and drawbacks of the known dosage forms.

### SUMMARY OF THE INVENTION

One aspect of the invention is directed towards a ready-to-use pharmaceutical composition for preparation of an injectable comprising a gemcitabine solution concentrate in a mixture of water and at least one additional physiologically-acceptable solvent or solubilizer, wherein the solution has a gemcitabine concentration of about 16 mg/ml to about 110 mg/ml and a pH of about 3.5 to about 10.

In a preferred embodiment, the length of time t₉₅ during which 95% of the initial gemcitabine content remains after decomposition is greater than about 100 days at 25° C. Preferably, t₉₅ is greater than about 1000 days at 25° C. More preferably, t₉₅ is greater than about 2000 days at 25° C. Most preferably, t₉₅ is about 2300 days at 25° C.

In a preferred embodiment, the length of time t₉₅ during which 95% of the initial gemcitabine content remains after decomposition is greater than about 50 days at 40° C. Preferably, t₉₅ is about 600 days at 40° C.

In a preferred embodiment, the length of time t₉₅ during which 95% of the initial gemcitabine content remains after decomposition is greater than about 50 days at 60° C. Preferably, t₉₅ is about 150 days at 60° C.

In a preferred embodiment, the gemcitabine solution concentrate is not reconstituted from a solid substance at least 24 hours before being administered to a mammal. Preferably, the gemcitabine solution concentrate is not reconstituted from a solid substance at least 72 hours before being administered to a mammal.

In a preferred embodiment, the physiologically-acceptable solvent is selected from the group consisting of ethyl alcohol, polyethylene glycol 200-600, 1,2-propanediol (propylene glycol), and mixtures thereof. Preferably, the physiologically-acceptable solvent is ethyl alcohol.

In a preferred embodiment, the ethyl alcohol is in the amount of about 20% to about 90% by volume. Preferably, the ethyl alcohol is in the amount of about 50% by volume. Also preferably, the ethyl alcohol is in the amount of about 60% by volume.

In a preferred embodiment, the physiologically-acceptable solubilizer is urea.

In a preferred embodiment, the gemcitabine concentration is about 20 mg/ml to about 90 mg/ml. Preferably, the gemcitabine concentration is about 80 mg/ml.

In a preferred embodiment, the gemcitabine concentration is about 40 mg/ml to about 60 mg/ml. Preferably, the gemcitabine concentration is about 50 mg/ml.

In a preferred embodiment, the solution concentrate has a pH of about 5 to about 10. Preferably, the solution concentrate has a pH of about 7 to about 8.

In a preferred embodiment, the pH of the solution concentrate is adjusted by combining or converting gemcitabine base with/to a physiologically-acceptable acid addition salt thereof. Preferably, the physiologically-acceptable acid addition salt is gemcitabine hydrochloride.

In a preferred embodiment, the pH of the solution concentrate is adjusted with at least one physiologically-acceptable acid. Preferably, the acid is selected from the group consisting of hydrochloric acid, phosphoric acid, sulfuric acid, acetic acid, lactic acid, citric acid, methanesulfonic acid, and ethanesulfonic acid. More preferably, the acid is hydrochloric acid.

In a preferred embodiment, the pH of the solution concentrate is adjusted with at least one physiologically-acceptable base. Preferably, the base is selected from the group consisting of sodium hydroxide, potassium hydroxide, calcium hydroxide, and magnesium hydroxide. More preferably, the base is sodium hydroxide.

In a preferred embodiment, the pH of the solution concentrate is adjusted with a buffer. Preferably, at least one functional group of the buffer's acid or base is within the pK range about 2.5 to about 11.

Preferably, the buffer is prepared from a reagent selected from the group consisting of tris(hydroxymethyl)-aminomethane, 1-deoxy-(methylamino)-D-glucitol, sodium acetate, disodium hydrogen phosphate, and mixtures thereof.

Preferably, the buffer is in the amount of about 0.001 g to about 100 g buffer component per 1 g of gemcitabine. Preferably, the buffer is in the amount of about 0.05 g to about 20 g buffer component per 1 g of gemcitabine. More preferably, the buffer is in the amount of about 0.1 g to about 10 g buffer component per 1 g of gemcitabine.

In a preferred embodiment, the pharmaceutical composition according to the invention further comprises at least one tonic adjuvant, preservative, antioxidant, or mixtures thereof.

Another aspect of the invention is directed towards a package for distribution comprising the pharmaceutical composition according the invention, wherein the solution is diluted for administration to a mammal without further solubilization of gemcitabine.

Another aspect of the invention is directed towards a method of parenteral administration to a mammal comprising administering the pharmaceutical composition according to the invention to a mammal in need thereof.

Another aspect of the invention is directed towards a method of treating neoplastic disease in a mammal comprising administering the pharmaceutical composition according to the invention to a mammal in need thereof.

### DETAILED DESCRIPTION OF THE INVENTION

The term “ready-to-use” means that the solution referred to is not reconstituted from a solid, such as, for example, from a crystalline or amorphous solid or a lyophilisate immediately before its administration to a mammal.

The term “solution concentrate” refers to gemcitabine solution concentrates prepared according to the invention.

In the experiments underlying the invention it was found that the solubility of gemcitabine in aqueous solution concentrates can be significantly increased at high pH levels by dissolving the gemcitabine in a mixture of water and at least one other physiologically-acceptable solvent or solubilization agent.

In the experiments underlying the invention it was also found that gemcitabine solution concentrates according to the invention with a pH of about 3.5 to about 10 exhibited high storage stability rates at various storage temperatures.

Preferably, the solution concentrate according to the invention has a gemcitabine concentration of 50 mg/ml or 80 mg/ml, an ethyl alcohol proportion of 50 percent by volume or 60 percent by volume, and a pH of about 8.

The solution concentrates according to the invention have a gemcitabine concentration of about 16 mg to about 110 mg gemcitabine per ml solvent. Preferably, the gemcitabine concentration is about 50 mg/ml or about 80 mg/ml.

The solution concentrates according to the invention may contain the free gemcitabine base or a physiologically-acceptable acid addition salt thereof. Preferably used for the preparation of the solution concentrates according to the invention is the free gemcitabine base, more preferably the acid addition salt with an inorganic acid, and most preferably gemcitabine hydrochloride.

Suitable solvents for the solution concentrates according to the invention are, for example, mixtures of water with ethyl alcohol, glycerine, 1,2-propanediol (propylene glycol), polyethylene glycol 200-600, benzyl alcohol, trimethylene glycol, 1,3-butylene glycol, 2,3-butylene glycol, ethyl acetate, ethyl lactate, glycofurol (tetraglycol), solketal, physiologically acceptable cyclodextrines (α-, β-, γ-cyclodextrines), as well as their alkyl- and/or aryl-substituted derivatives and/or urea as solvent or tonic adjuvants. Preferably, a mixture of water, ethyl alcohol, polyethylene glycol 200-600 and/or 1,2-propanediol (propylene glycol) is used. More preferably, a mixture of water and ethyl alcohol and/or polyethylene glycol 300-400 is used. Even more preferably, a mixture of water and ethyl alcohol is used. The mixtures can be used in various proportions.

Preferably, the pH of the solution concentrates of the invention is about 3.5 to about 10. In one embodiment, the gemcitabine solution concentrates have a pH of about 4 to about 5. In another embodiment, the pH is about 5 to about 10, preferably about 7 to about 9. In a preferred embodiment, the pH of the solution concentrates matches the pH of physiological tissue and blood, which is about 7.35 to about 7.55. In a more preferred embodiment, the pH of the concentrate is about 7.8 to about 8.2.

Adjustment of the pH of a solution concentrate according to the invention can be effected by mixing a suitable proportion of the gemcitabine base with a physiologically-acceptable acid addition salt thereof. Preferably, gemcitabine hydrochloride is used.

According to the invention, it is also possible to adjust the pH with at least one physiologically-acceptable acidifier and/or alkalizer. Suitable for this purpose are, for example, inorganic acids and bases, such as hydrochloric acid, sulfuric acid, sulfurous acid, nitric acid, nitrous acid, phosphoric acid, phosphorous acid, carbonic acid, sodium hydroxide, potassium hydroxide, calcium hydroxide and magnesium hydroxide; alkaline salts and alkaline-earth salts, as well as alkaline hydrogen salts and alkaline-earth hydrogen salts of the inorganic oxo acids of phosphorus, sulfur, carbon and nitrogen, such as, e.g., sodium phosphate and its hydrates, sodium dihydrogen phosphate and its hydrates, disodium hydrogen phosphate and its hydrates, disodium sulfate, sodium hydrogen sulfate, sodium sulfite, calcium sulfite, magnesium sulfite, calcium hydrogen carbonate, sodium carbonate, sodium hydrogen carbonate, sodium nitrate, sodium nitrite, calcium nitrite, magnesium nitrate and magnesium nitrite; chlorine salts such as, e.g., sodium chloride, calcium chloride, and magnesium chloride; organic bases and acids, as well as the alkaline and alkaline-earth salts thereof, including, e.g., formic acid, acetic acid, propionic acid, lactic acid, oxalic acid, malonic acid, maleic acid, tartaric acid, citric acid, pyruvic acid, benzoic acid, methanesulfonic acid, ethanesulfonic acid, p-toluenesulfonic acid, salicylic acid, ascorbic acid, tris(hydroxymethyl)aminomethane (2-amino-2-hydroxymethyl-1,3-propanediol); trometamol; TRIS), 1-deoxy-(methylamino)-D-glucitol (N-methylglucamine, meglumine); alkali salts and alkaline-earth salts of organic bases and acids, such as, for example, sodium acetate; and mixtures thereof.

Preferably, the pH of the solution concentrates is adjusted with hydrochloric acid, phosphoric acid, sulfuric acid, sodium hydroxide, sodium phosphate and their hydrates, sodium hydrogen phosphate and its hydrates, disodium hydrogen phosphate and its hydrates, acetic acid, lactic acid, citric acid, methanesulfonic acid, ethanesulfonic acid, tris(hydroxymethyl)aminomethane (trometamol; TRIS), or 1-deoxy-(methylamino)-D-glucitol (N-methylglucamine, meglumine). More preferably, sodium hydroxide, hydrochloric acid, tris(hydroxymethyl)aminomethane (trometamol; TRIS), or 1-deoxy-(methylamino)-D-glucitol (N-methylglucamine, meglumine) is used.

The pH can also be adjusted and/or stabilized by a pH-buffer comprising at least one physiologically-acceptable acidifier and/or alkalizer. Preferably, buffer systems where the pK of at least one functional group of the buffer base or buffer acid forming the buffer lies within the pK range of 2.49 to 11.01 are used. More preferred buffer systems are sodium acetate, tris(hydroxymethyl)aminomethane (trometamol; TRIS), 1-deoxy-(methylamino)-D-glucitol (N-methylglucamine, meglumine), disodium hydrogen phosphate, and mixtures thereof.

The buffer concentration in the solution concentrates according to the invention is about 0.001 g to about 100 g buffer component to 1 g gemcitabine, preferably about 0.05 g to about 20 g buffer component to 1 g gemcitabine, and more preferably about 0.1 g to about 10 g buffer component to 1 g gemcitabine.

The solution concentrates according to the invention may optionally contain, in addition, at least one tonic adjuvant, at least one preservative, and/or at least one antioxidant.

For example, the following can be used as tonic agents and solubilization agents for the solution concentrates according to the invention: physiologically-acceptable inorganic alkali or alkaline-earth salts, such as, e.g., sodium chloride, calcium chloride, magnesium chloride, sodium sulfate, sodium carbonate and calcium hydrogen carbonate; physiologically acceptable organic salts, such as, e.g., sodium lactate; physiologically acceptable carbohydrates, such as, e.g., physiologically acceptable cyclodextrins (α-, β-, γ-cyclodextrins), as well as their alkyl-substituted and/or aryl-substituted derivatives, glucose, fructose, sorbitol, mannitol, galactose, inositol, maltitol, lactose, trehalose, maltose, sucrose, dextran 1, dextran 10, dextran 40, dextran 70, starch and hydroxyethyl starch; physiologically acceptable amino acids, peptides or proteins, such as, say, glycine, albumin and gelatins, and mixtures thereof.

Preferred tonic agents are sodium chloride, calcium chloride, glucose, mannitol, and lactose. More preferred tonic agents are sodium chloride, glucose, and mannitol.

The following, for example, can be used as preservatives for the solution concentrates according to the invention: chlorocresol, benzyl alcohol, p-hydroxybenzoic acid ester, as well as, e.g., propylparaben, ethylparaben, and methylparaben. Preferred preservatives are benzyl alcohol, propylparaben, and methylparaben. More preferably, benzyl alcohol is used.

The following, for example, can be used as antioxidants for the solutions according to the invention: oxygen-reducing antioxidants, such as, e.g., sodium metabisulfite and sodium sulfite, or transition-metal-ion binding antioxidants and complexing (chelating) antioxidants, such as, e.g., sodium edetate.

To prepare an infusion solution, the solution concentrate according to the invention is diluted with the required amount of a carrier solution, such as, e.g., 0.9% sodium chloride solution or 5% glucose solution.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Examples 1-2

To determine the stability gemcitabine solutions (Example 1) derived from dry substance known from the related art (Journal of Pharmaceutical Sciences 2000, 89(7), 885-891), aqueous solutions (a concentration of 40 mg/ml of gemcitabine, pH 3.2; 3.25 mg/ml of sodium acetate; hydrochloric acid, sodium hydroxide) were prepared, and the content of active substance determined on a time-resolved basis by HPLC. This composition is approximately comparable to a conventional reconstituted gemcitabine concentrate reconstituted as prescribed from GEMZAR® lyophilisate.

According to the technical information about the product GEMZAR® lyophilisate, its reconstitution yields solutions with a pH in the range of 2.7-3.3. Known from the state of the art is that these reconstituted solutions were not suitable for storage but had to be administered to the patient at once. The following examples show that the gemcitabine solution concentrates according to the invention possess considerably improved storage stability and accordingly permit the use of ready-to-use solutions in therapeutic practice.

From the stability data obtained by means of HPLC, it was possible to regressively interpolate for each of the storage temperatures indicated the length of time t₉₅ during which a pharmaceutically required minimum content of 95% of the initial content of active substance is obtained following decomposition.

TABLE 1
Comparison of the stability of a gemcitabine solution known from the
related art (Example 1) with an exemplary gemcitabine solution
according to the invention (Example 2)

Example:

| | | 1 | 2 |
|---|---|---|---|
| Gemcitabine (mg/ml) | | 40 | 40 |
| 1N Sodium hydroxide/1N Hydrochloric acid | | q.s. | q.s. |
| Sodium acetate (mg/ml) | | 3.25 | — |
| Ethyl alcohol (percent by volume) | | — | 50 |
| Water for injection | | q.s. | q.s. |
| pH | | 3.2 | 8.2 |
| 25° C. | Start | 100.0 | 100.0 |
| Content (rel. %) | 3 mo. | 95.38 | 99.70 |
| | 6 mo. | 83.70 | 99.61 |
| t₉₅ (days; 25° C.) | | 68 | 2292 |
| 40° C. | Start | 100.0 | 100.0 |
| Content (rel. %) | 1 mo. | 89.41 | 99.77 |
| | 2 mo. | 78.30 | 99.51 |
| | 3 mo. | 62.05 | 99.28 |
| t₉₅ (days; 40° C.) | | 15 | 620 |
| 60° C. | Start | 100.0 | 100.0 |
| Content (rel. %) | 1 wk. | 79.21 | 99.72 |
| | 2 wk. | 56.06 | 99.51 |
| | 3 wk. | 20.05 | 99.30 |

| | | |
|---|---|---|
| t₉₅ (days; 60° C.) | 2.2 | 151 |

(mo. = 30 days; wk. = 7 days)

A comparison of the t₉₅ values in Table 1 clearly indicates the efficiency of the present invention. Thus, for example, by changing the pH from about 3 to about 8, the stability is increased 30-fold to 70-fold compared to the related art (see above publication).

### Example 3

In order to determine the saturation concentration of gemcitabine in aqueous ethyl alcohol solutions, gemcitabine solutions with various volume percentages of ethyl alcohol were prepared and analyzed.

Gemcitabine hydrochloride was placed beforehand in the mixing vessel, reacted with a stoichiometric amount of alkalizer (1N sodium hydroxide) dissolved in water, then treated each time with 80% of the required amounts of water for injection as determined in preliminary tests as well as with the amounts of ethyl alcohol required each time.

The components contained in the batch were subsequently dissolved at room temperature (19-23° C.) while agitating for 6 hours such that an undissolved sediment of gemcitabine hydrochloride remained in the mixing vessel. The pH of the batch was determined potentiometrically and, when required, adjusted to a pH of 10 with 1N sodium hydroxide or 1N hydrochloric acid, so as to achieve at this pH a complete conversion of the gemcitabine hydrochloride to the dissolved gemcitabine base.

The batch was stored for 24 hours at 19-23° C. The pH was then checked and only those clear supernatant fluids of the batches were further processed which had a pH of 10±0.5. These batches were then filtered until free of particles, and the gemcitabine content was determined by HPLC. The results of these tests are listed in Table 2 below.

TABLE 2
Gemcitabine saturation concentrations of aqueous ethyl alcohol
solutions (at 19-23° C. and pH 9.5-10.5)

| | Proportion of | Content of | Content of |
|---|---|---|---|
| | ethyl alcohol | gemcitabine | gemcitabine |
| | (percent by | HCl | base |
| Test solution | volume) | [mg/ml] | [mg/ml] |
| 1 | 0 | 15.81 | 13.9 |
| 2 | 10 | 21.92 | 19.3 |
| 3 | 20 | 26.26 | 23.1 |
| 4 | 30 | 33.67 | 29.6 |
| 5 | 40 | 39.22 | 34.5 |
| 6 | 50 | 49.38 | 43.4 |
| 7 | 60 | 57.39 | 50.4 |
| 8 | 70 | 55.28 | 48.6 |
| 9 | 80 | 48.30 | 42.4 |
| 10 | 90 | 31.21 | 27.4 |
| 11 | 100 | 5.35 | 4.70 |

Interpolation (polynomial of the 6th degree, R²>0.997) of the experimentally obtained data provided a maximum gemcitabine solubility of 50 mg/ml of gemcitabine, or 55 mg/ml of gemcitabine hydrochloride, with about 66 percent by volume of ethyl alcohol at a pH of 10±0.5.

### Example 4

The maximum solubility of gemcitabine hydrochloride was determined with further use of 1N sodium hydroxide, water for injection and absolute ethyl alcohol in a proportion of 66 percent by volume recognized as optimum for that purpose, at increased solution temperatures (35-45° C. for 6 hours under agitation) and subsequent cooling to room temperature (19-23° C.), and then stored for 24 hours at 19-23° C. A gemcitabine content of 95.1 mg/ml, or 108.3 mg/ml of gemcitabine hydrochloride, was determined by HPLC in the particle-free supernatant of the stored solutions.

### Example 5

The stability of gemcitabine in aqueous ethyl alcohol solution concentrates was determined with gemcitabine solution concentrates having the composition characteristics listed below (test solutions 12-21). The “stability of the solution” is always to be understood as the stability of the gemcitabine in solution, i.e., as the long-range constancy of the concentration of the starting compound after being dissolved.

To prepare solutions listed in Table 3, water for injection was changed in advance with 90% of the required amount, and absolute ethyl alcohol was added as an additional solvent, with 90% of the required amount. The batch was heated to 35-45° C. and 95% of the required amount of corresponding alkalizer (for example, 1N sodium hydroxide) was added. The batch was then heated to 35-45° C.

The gemcitabine hydrochloride was added at 100% of the required amount and, while agitating, dissolved until clear, whereupon the pH was potentiometrically determined and adjusted with the required amount of corresponding alkalizer (for example, 1N sodium hydroxide) to the desired level.

The batch was filled to the mark with the required amount of water for injection and the additional solvent (absolute ethyl alcohol). The batch, heated to 35-45° C., was agitated until a clear solution was obtained.

The pH was again verified and, when necessary, adjusted to the desired level with sodium hydroxide or hydrochloric acid.

The batch was cooled to room temperature (19-23° C.) and sterilized by filtration in a sterile receiving flask.

The solution concentrate obtained was aseptically filled into perforable stopper vials (5 ml per vial), which were sealed with perforable stoppers and crimped. The perforable stopper vials were stored, protected from light, at 25° C., and “under accelerated conditions” at 40° C.

For each test procedure, samples were subjected to analysis (by HPLC) for content and purity, and the pH was (potentiometrically) determined.

The results of the analysis of the stability procedure test samples are listed in Table 3.

TABLE 3
Stability and composition of the test solutions 12-21

| Test solution | | 12 | 13 | 14 | 15 | 16 | 17 | 18 | 19 | 20 | 21 |
|---|---|---|---|---|---|---|---|---|---|---|---|
| Gemcitabine | | 50 | 50 | 80 | 40 | 30 | 40 | 30 | 25 | 80 | 60 |
| (mg/ml) | | | | | | | | | | | |
| 1N Sodium hydroxide/ | | — | q.s. | q.s. | — | — | q.s. | q.s. | q.s. | q.s. | q.s. |
| 1N Hydrochloric acid | | | | | | | | | | | |
| Disodium hydrogen | | — | — | — | — | — | — | — | — | — | — |
| phosphate (H₂O)₂ | | | | | | | | | | | |
| (mg/ml) | | | | | | | | | | | |
| 5N Trometamol | | — | — | — | q.s. | — | — | — | — | — | — |
| 5N Meglumine | | q.s. | — | — | — | q.s. | — | — | — | — | — |
| Ethyl alcohol | | 50 | 50 | 60 | 40 | 50 | 50 | 50 | 40 | 66 | 50 |
| (vol. %) | | | | | | | | | | | |
| Water for injection | | q.s. | q.s. | q.s. | q.s. | q.s. | q.s. | q.s. | q.s. | q.s. | q.s. |
| pH | | 7.0 | 8.4 | 8.0 | 8.9 | 8.0 | 8.2 | 8.1 | 8.8 | 7.6 | 8.4 |
| 25° C. | Start | 100.0 | 100.0 | 100.0 | 100.0 | 100.0 | 100.0 | 100.0 | 100.0 | 100.0 | 100.0 |
| Content (rel. %) | 3 mo. | 99.83 | 99.70 | 99.51 | 99.64 | 99.44 | 99.54 | 99.57 | 99.84 | 99.83 | 99.85 |
| | 6 mo. | 99.59 | 99.61 | 99.54 | 99.34 | 99.78 | 99.24 | 99.34 | 99.51 | 99.67 | 99.70 |
| | 9 mo. | 99.60 | 99.62 | 99.63 | 99.18 | 99.68 | 99.63 | 99.62 | 99.51 | 99.49 | 99.55 |
| | 12. mo. | 99.47 | 99.51 | 99.36 | 99.10 | 98.57 | 99.52 | 99.49 | 99.40 | 99.33 | 99.43 |
| 40° C. | Start | 100.0 | 100.0 | 100.0 | 100.0 | 100.0 | 100.0 | 100.0 | 100.0 | 100.0 | 100.0 |
| Content (rel. %) | 3 mo. | 99.41 | 99.02 | 99.11 | 99.59 | 98.81 | 99.28 | 98.54 | 98.82 | 98.82 | 98.95 |
| | 6 mo. | 98.60 | 99.21 | 99.04 | 98.74 | 97.58 | 98.67 | 97.25 | 97.63 | 98.05 | 97.69 |
| | 9 mo. | 97.05 | 96.33 | 96.74 | 95.11 | 96.48 | 97.83 | 95.81 | 96.59 | 96.87 | 96.79 |
| | 12 mo. | 95.83 | 94.98 | 95.52 | 93.65 | 95.32 | 97.28 | 94.30 | 95.44 | 95.99 | 95.52 |

The results of the stability studies of aqueous ethyl alcohol gemcitabine solution concentrates show that gemcitabine in the aqueous ethyl alcohol concentrates has a high storage stability.

### Example 6

To determine the storage temperature-dependent stability of buffered gemcitabine solution concentrates, gemcitabine solution concentrates were prepared with disodium hydrogen phosphate dihydrate-buffered compositions (test solutions 22-27) described in Example 3 and analyzed. The results of these studies are listed in Table 4.

TABLE 4
Stability of phosphate-buffered test solutions 22-27

| Test solution | | 22 | 23 | 24 | 25 | 26 | 27 |
|---|---|---|---|---|---|---|---|
| Gemcitabine (mg/ml) | | 30 | 25 | 50 | 25 | 50 | 60 |
| 1N Sodium hydroxide/ | | q.s. | q.s. | q.s. | q.s. | q.s. | q.s. |
| 1N Hydrochloric acid | | | | | | | |
| Disodium hydrogen | | 2 | 2 | 2 | 4 | 4 | 4 |
| phosphate.(H₂O)₂ (mg/ml) | | | | | | | |
| 5N Trometamol | | — | — | — | — | — | — |
| 5N Meglumine | | — | — | — | — | — | — |
| Ethyl alcohol (vol. %) | | 50 | 50 | 50 | 40 | 66 | 50 |
| Water for injection | | q.s. | q.s. | q.s. | q.s. | q.s. | q.s. |
| pH | | 7.5 | 7.2 | 7.1 | 7.8 | 7.6 | 7.4 |
| 25° C. | Start | 100.0 | 100.0 | 100.0 | 100.0 | 100.0 | 100.0 |
| Content (rel. %) | 3 mo. | 99.54 | 99.63 | 99.47 | 99.74 | 99.87 | 99.89 |
| | 6 mo. | 99.82 | 99.34 | 99.34 | 99.61 | 99.57 | 99.71 |
| | 9 mo. | 99.63 | 99.53 | 99.22 | 99.54 | 99.59 | 99.45 |
| | 12 mos. | 98.51 | 99.42 | 99.19 | 99.13 | 99.23 | 99.53 |
| 40° C. | Start | 100.0 | 100.0 | 100.0 | 100.0 | 100.0 | 100.0 |
| Content (rel. %) | 3 mo. | 98.71 | 99.28 | 98.58 | 98.72 | 98.73 | 98.65 |
| | 6 mo. | 97.68 | 98.56 | 97.35 | 97.53 | 98.15 | 97.67 |
| | 9 mo. | 96.53 | 97.73 | 95.71 | 96.79 | 96.59 | 96.73 |
| | 12 mo. | 95.42 | 96.68 | 94.40 | 95.94 | 96.11 | 95.62 |

As indicated in Table 4, even the buffered solution concentrates remained clear and free of particles without crystallization during the period of storage. The results of stability studies carried out at storage temperatures of 25° C. and 40° C. show that both unbuffered and buffered solution concentrates according to the invention are stable even at storage temperatures of 40° C.

### Examples 7-34

Aqueous solution concentrates with compositions listed in Table 5 were prepared.

TABLE 5
Examples of gemcitabine solution concentrates according to the invention

| | Gemcitabine | Additional | | Alkalizers/ | Additional |
|---|---|---|---|---|---|
| | Content | Solvents (vol %) or | | Acidifiers or | Adjuvants |
| Example | (mg/ml) | Solubilizers (m/V) | pH | Buffers (m/V) | (m/V) |
| 7 | 20.0 | 30 vol. % PEG 400 | 8.2 | Sodium hydroxide/ | |
| | | | | Hydrochloric acid | |
| 8 | 20.0 | 40 vol. % PEG 400 | 7.6 | Sodium hydroxide/ | 0.13% (m/V) |
| | | | | Hydrochloric acid | Methylparaben, |
| | | | | | 0.02% (m/V) |
| | | | | | Propylparaben |
| 9 | 20.0 | 30 vol. % PEG 400 | 7.0 | Sodium hydroxide/ | |
| | | | | Hydrochloric acid | |
| 10 | 20.0 | 40% (m/V) urea | 7.0 | Sodium hydroxide/ | |
| | | | | Hydrochloric acid | |
| 11 | 20.0 | 40% (m/V) urea | 7.2 | Sodium hydroxide/ | |
| | | | | Hydrochloric acid | |
| | | | | 0.02% (m/V) | |
| | | | | Sodium dihydrogen | |
| | | | | phosphate | |
| 12 | 30.0 | 40 vol. % | 10.0 | Sodium hydroxide/ | |
| | | ethyl alcohol | | Hydrochloric acid | |
| 13 | 30.0 | 40 vol. % | 10.0 | Sodium hydroxide/ | |
| | | ethyl alcohol | | Hydrochloric acid | |
| | | | | 0.1% (m/V) | |
| | | | | Meglumine | |
| 14 | 30.0 | 40 vol. % | 9.0 | Sodium hydroxide/ | |
| | | ethyl alcohol | | Hydrochloric acid | |
| 15 | 30.0 | 40 vol. % | 9.0 | Sodium hydroxide/ | |
| | | ethyl alcohol | | Hydrochloric acid | |
| | | | | 0.1% (m/V) | |
| | | | | Meglumine | |
| 16 | 30.00 | 40 vol. % | 8.0 | Sodium hydroxide/ | |
| | | ethyl alcohol | | Hydrochloric acid | |
| 17 | 30.0 | 40 vol. % | 8.3 | Sodium hydroxide/ | |
| | | ethyl alcohol | | Hydrochloric acid | |
| | | | | 0.1% (m/V) TRIS | |
| 18 | 30.0 | 40 vol. % | 7.6 | Sodium hydroxide/ | |
| | | ethyl alcohol | | Hydrochloric acid | |
| | | | | 0.1% (m/V) TRIS | |
| 19 | 20.0 | 50 vol. % | 5.1 | Sodium hydroxide/ | |
| | | ethyl alcohol | | Hydrochloric acid | |
| | | | | 0.025% (m/V) | |
| | | | | Sodium acetate | |
| 20 | 40.0 | 50 vol. % | 6.0 | Sodium hydroxide/ | |
| | | ethyl alcohol | | Hydrochloric acid | |
| 21 | 40.0 | 50 vol. % | 8.0 | Sodium hydroxide/ | |
| | | ethyl alcohol | | Hydrochloric acid | |
| 22 | 50.0 | 50 vol. % | 9.1 | Sodium hydroxide/ | |
| | | ethyl alcohol | | Hydrochloric acid | |
| 23 | 50.0 | 50 vol. % | 7.5 | Sodium hydroxide/ | |
| | | ethyl alcohol | | Hydrochloric acid | |
| 24 | 50.0 | 50 vol. % | 6.8 | Sodium hydroxide/ | |
| | | ethyl alcohol | | Hydrochloric acid | |
| 25 | 50.0 | 50 vol. % | 6.8 | Sodium hydroxide/ | 1.0% (m/V) |
| | | ethyl alcohol | | Hydrochloric acid | Benzyl alcohol |
| 26 | 20.0 | 60 vol. % | 5.2 | Sodium hydroxide/ | |
| | | ethyl alcohol | | Hydrochloric acid | |
| | | | | 0.025% (m/V) | |
| | | | | Sodium acetate | |
| 27 | 20.0 | 60 vol. % | 5.2 | Sodium hydroxide/ | 1.0% (m/V) |
| | | ethyl alcohol | | Hydrochloric acid | Benzyl alcohol |
| | | | | 0.025% (m/V) | |
| | | | | Sodium acetate | |
| 28 | 80.0 | 60 vol. % | 8.8 | Sodium hydroxide/ | |
| | | ethyl alcohol | | Hydrochloric acid | |
| | | | | 0.1% (m/V) TRIS | |
| 29 | 80.0 | 60 vol. % | 7.8 | Sodium hydroxide/ | |
| | | ethyl alcohol | | Hydrochloric acid | |
| 30 | 80.0 | 60 vol. % | 7.8 | Sodium hydroxide/ | 0.13% (m/V) |
| | | ethyl alcohol | | Hydrochloric acid | Methylparaben |
| | | | | | 0.02% (m/V) |
| | | | | | Propylparaben |
| 31 | 80.0 | 60 vol. % | 8.9 | Sodium hydroxide/ | |
| | | ethyl alcohol | | Hydrochloric acid | |
| 32 | 20.0 | 30 vol. % | 3.5 | Sodium hydroxide/ | |
| | | ethyl alcohol | | Hydrochloric acid | |
| | | | | 0.025% (m/V) | |
| | | | | Sodium lactate | |
| 33 | 20.0 | 30 vol. % | 4.0 | Sodium hydroxide/ | |
| | | ethyl alcohol | | Hydrochloric acid | |
| | | | | 0.025% (m/V) | |
| | | | | Sodium lactate | |
| 34 | 20.0 | 30 vol. % | 4.0 | Sodium hydroxide/ | |
| | | ethyl alcohol | | Hydrochloric acid | |
| | | | | 0.025% (m/V) | |
| | | | | Sodium acetate | |

m/V = mass/volume = mg/ml
vol % = (volume − additive)/(volume − solvent) = ml/ml

What is claimed is:

1. A ready-to-use pharmaceutical composition for preparation of an injectable comprising a gemcitabine solution concentrate in a mixture of water and at least one additional physiologically-acceptable solvent or solubilizer, wherein the solution has a gemcitabine concentration of about 16 mg/ml to about 110 mg/ml and a pH of about 3.5 to about 10.

2. The pharmaceutical composition according to claim 1, wherein the length of time t₉₅, after which 95% of the initial gemcitabine content is remaining, is greater than about 100 days at 25° C.

3. The pharmaceutical composition according to claim 2, wherein the length of time t₉₅, after which 95% of the initial gemcitabine content is remaining, is greater than about 1000 days at 25° C.

4. The pharmaceutical composition according to claim 3, wherein the length of time t₉₅, after which 95% of the initial gemcitabine content is remaining, is greater than about 2000 days at 25° C.

5. The pharmaceutical composition according to claim 4, wherein the length of time t₉₅, after which 95% of the initial gemcitabine content is remaining, is about 2300 days at 25° C.

6. The pharmaceutical composition according to claim 1, wherein the length of time t₉₅, after which 95% of the initial gemcitabine content is remaining, is greater than about 50 days at 40° C.

7. The pharmaceutical composition according to claim 6, wherein the length of time t₉₅, after which 95% of the initial gemcitabine content is remaining is about 600 days at 40° C.

8. The pharmaceutical composition according to claim 1, wherein the length of time t₉₅, after which 95% of the initial gemcitabine content is remaining, is greater than about 50 days at 60° C.

9. The pharmaceutical composition according to claim 8, wherein the length of time t₉₅, after which 95% of the initial gemcitabine content is remaining is about 150 days at 60° C.

10. The pharmaceutical composition according to claim 1, wherein the gemcitabine solution concentrate is not reconstituted from a solid substance within at least 24 hours before being administered to a mammal.

11. The pharmaceutical composition according to claim 10, wherein the gemcitabine solution concentrate is not reconstituted from a solid substance within at least 72 hours before being administered to a mammal.

12. The pharmaceutical composition according to claim 1, wherein the physiologically-acceptable solvent is selected from the group consisting of ethyl alcohol, polyethylene glycol 200-600, 1,2-propanediol (propylene glycol), and mixtures thereof.

13. The pharmaceutical composition according to claim 12, wherein the physiologically-acceptable solvent is ethyl alcohol.

14. The pharmaceutical composition according to claim 13, wherein the ethyl alcohol is in the amount of about 20% to about 90% by volume.

15. The pharmaceutical composition according to claim 14, wherein the ethyl alcohol is in the amount of about 50% by volume.

16. The pharmaceutical composition according to claim 14, wherein the ethyl alcohol is in the amount of about 60% by volume.

17. The pharmaceutical composition according to claim 1, wherein the physiologically-acceptable solubilizer is urea.

18. The pharmaceutical composition according to claim 1, wherein the gemcitabine concentration is about 20 mg/ml to about 90 mg/ml.

19. The pharmaceutical composition according to claim 18, wherein the gemcitabine concentration is about 80 mg/ml.

20. The pharmaceutical composition according to claim 1, wherein the gemcitabine concentration is about 40 mg/ml to about 60 mg/ml.

21. The pharmaceutical composition according to claim 20, wherein the gemcitabine concentration is about 50 mg/ml.

22. The pharmaceutical composition according to claim 1, wherein the solution has a pH of about 5 to about 10.

23. The pharmaceutical composition according to claim 22, wherein the solution has a pH of about 7 to about 8.

24. The pharmaceutical composition according to claim 1, wherein the pH of the concentrate is adjusted by combining gemcitabine base with a physiologically-acceptable acid addition salt thereof.

25. The pharmaceutical composition according to claim 24, wherein the physiologically-acceptable acid addition salt is gemcitabine hydrochloride.

26. The pharmaceutical composition according to claim 1, wherein the pH of the concentrate is adjusted with at least one physiologically-acceptable acid.

27. The pharmaceutical composition according to claim 26, wherein the acid is selected from the group consisting of hydrochloric acid, phosphoric acid, sulfuric acid, acetic acid, lactic acid, citric acid, methanesulfonic acid, and ethanesulfonic acid.

28. The pharmaceutical composition according to claim 27, wherein the acid is hydrochloric acid.

29. The pharmaceutical composition according to claim 1, wherein the pH of the concentrate is adjusted with at least one physiologically-acceptable base.

30. The pharmaceutical composition according to claim 29, wherein the base is selected from the group consisting of sodium hydroxide, potassium hydroxide, calcium hydroxide, and magnesium hydroxide.

31. The pharmaceutical composition according to claim 30, wherein the base is sodium hydroxide.

32. The pharmaceutical composition according to claim 1, wherein the pH of the concentrate is adjusted with a buffer.

33. The pharmaceutical composition according to claim 32, wherein at least one functional group of the buffer's acid or base is within the pK range from about 2.5 to about 11.

34. The pharmaceutical composition according to claim 32, wherein the buffer is prepared from a reagent selected from the group consisting of tris(hydroxymethyl)-aminomethane, 1-deoxy-(methylamino)-D-glucitol, sodium acetate, disodium hydrogen phosphate, and mixtures thereof.

35. The pharmaceutical composition according to claim 32, wherein the buffer is in the amount of about 0.001 g to about 100 g buffer component per 1 g of gemcitabine.

36. The pharmaceutical composition according to claim 35, wherein the buffer is in the amount of about 0.05 g to about 20 g buffer component per 1 g of gemcitabine.

37. The pharmaceutical composition according to claim 36, wherein the buffer is in the amount of about 0.1 g to about 10 g buffer component per 1 g of gemcitabine.

38. The pharmaceutical composition according to claim 1, further comprising at least one tonic adjuvant, preservative, antioxidant, or mixtures thereof.

39. A package for distribution comprising the pharmaceutical composition according to claim 1, wherein the solution is diluted for administration to a mammal without further solubilization of gemcitabine.

40. A method of parenteral administration to a mammal comprising administering the pharmaceutical composition of claim 1 to a mammal in need thereof.

41. A method of treating neoplastic disease in a mammal comprising administering the pharmaceutical composition of claim 1 to a mammal in need thereof.
